(19) United States
(12) Patent Application Publication
(10) Pub. No.: US 2025/0059554 A1
(43) Pub. Date: Feb. 20, 2025

(54) GENE KWE2 FOR REGULATING CORN EAR GRAIN WEIGHT AND YIELD, ENCODED PROTEIN, INDEL1 MARKER, EXPRESSION VECTOR AND USE THEREOF IN PLANT TRAIT IMPROVEMENT

(71) Applicant: HENAN AGRICULTURAL UNIVERSITY, Zhengzhou (CN)

(72) Inventors: Jihua TANG, Zhengzhou (CN); Zhiyuan FU, Zhengzhou (CN); Dong DING, Zhengzhou (CN); Weihua LI, Zhengzhou (CN); Yafei WANG, Zhengzhou (CN); Jun ZONG, Zhengzhou (CN)

(21) Appl. No.: 18/800,014

(22) Filed: Aug. 10, 2024

Related U.S. Application Data
(63) Continuation of application No. PCT/CN2023/093126, filed on May 10, 2023.

(30) Foreign Application Priority Data
May 14, 2022 (CN) 202210524175.7

(51) Int. Cl. C12N 15/82; C07K 14/415; C12Q 1/6895

(52) U.S. Cl. CPC C12N 15/8261; C07K 14/415; C12Q 1/6895; C12Q 2600/13; C12Q 2600/156

(57) ABSTRACT

Provided are a gene KWE2 for regulating corn car grain weight and yield, a protein encoding same, and an expression vector thereof. Further provided are an InDel1 marker of the gene KWE2 for regulating corn car grain weight and yield, and the use thereof in plant trait improvement.

### CROSS-REFERENCES TO RELATED APPLICATIONS

The present application is a continuation application of International Application No. PCT/CN2023/093126, filed on May 10, 2023, which claims priority to Chinese Patent Application No. 202210524175.7, filed on May 14, 2022. The disclosures of the above-mentioned applications are incorporated herein by reference in their entireties.

### REFERENCE TO SEQUENCE LISTING SUBMITTED ELECTRONICALLY

A Sequence Listing is provided herewith as a Sequence Listing XML, “SEQUENCE LISTING.xml” created on Nov. 6, 2024 and having a size of 21,496 bytes. The contents of the Sequence Listing XML are incorporated by reference herein in their entirety.

### TECHNICAL FIELD

The present application relates to the technical field of biological genetic engineering, and in particular, to a gene KWE2 for regulating corn ear grain weight and yield, an encoded protein, an InDel1 marker, an expression vector and a use thereof in improving plant traits.

### BACKGROUND

As the world's largest food crop, an important feed and industrial raw material, corn (Zea mays L.) plays an important role in ensuring food security in the world and in China (Gore et al., 2009; Prasanna 2012). In recent years, with the adjustment of China's agricultural production structure, the area planted with corn has been continuously decreasing. At the same time, due to the rapid development of the animal husbandry and deep processing industries, the consumer demand for corn has increased sharply, and the market demand has shown explosive growth. In particular, the recent COVID-19 epidemic and international turmoil have made food security issues increasingly prominent. In 2020, the average yield of corn in China is 421 kg/666.7 m², which is only 60% of the average yield of corn in the United States (735 kg/666.7 m²). There is still a lot of room for improvement. Therefore, with limited arable land, significantly increasing corn yield per unit area is the primary task to increase total output, get rid of dependence on imported corn as soon as possible, resolve the contradiction between corn supply and demand, and ensure food security.

The ear number per mu (1 mu˜0.0667 hectares), kernel number per ear, and 100-kernel weight are the three factors that constitute corn yield. Under a specific planting density, the kernel number per ear and hundred kernel weight determine the corn yield per unit area (Lopez-Reynoso and Hallauer 1998). The kernel number per ear is composed of kernel row number (KRN) and kernel number per row (KNR). The KRN genes that have been cloned and analyzed for their functions, such as KRN2 encoding the WD40 protein, negatively regulate the yield of corn and rice by synergizing with the unknown functional protein DUF1644, and have no negative effects on other agronomic traits (Chen et al., 2022). KRN4, located in the intergenic spacer region 60 Kb downstream of the SBP-box gene Unbranched3 (UB3), is a distal regulatory element of UB3, and its knockout mutant KRN and increases in an ear thickness (Liu et al., 2015). KRN1, which encodes an AP2 domain protein, is homologous to the key wheat domestication gene Q. The up-regulated expression of this gene increases the number of paired spikelet meristems and the KRN (Wang et al., 2019). KRN5 (Zm00001d016075) negatively regulates KNR, and also has a certain negative regulatory effect on the kernel number per ear and ear grain weight (An et al., 2022). The cloned ear length qEL7 gene encodes 1-aminocyclopropane-1-carboxylate oxidase 2 and negatively regulates spikelet number, floret fertility and ear length through ethylene signaling. Knocking out this gene can increase the panicle grain weight of hybrids by 13.4% (Ning et al., 2021). KNR6, which controls corn row kernel number, encodes a serine/threonine protein kinase and affects ear length and row kernel number by phosphorylating its interacting protein GTPase activating protein (AGAP). Deletion of the transposon in its promoter region can significantly increase yield (Jia et al., 2020). The candidate gene for ear length QTL (YIGE1) encodes an unknown protein that regulates corn yield by affecting the number of pistil florets. Overexpression of YIGE1 increases the size of the panicle inflorescence meristem, increases ear length and row grain number, thereby increasing com yield (Luo et al. al., 2022). The gene controlling the ear apical degeneration mutant (ear apical degeneration1, ead1) encodes an aluminum-activated malate efflux transporter protein. Overexpression of this gene can significantly increase ear length and row grain number (Pei et al., 2022).

Although the cloning and functional analysis of these genes provide important genetic resources for the breeding of new high-yield corn varieties, yield-related traits are all quantitative traits and are controlled by a few major-effect genes and multiple minor-effect genes. The functional genes currently obtained are very few: In order to elucidate the molecular mechanism of yield formation and provide excellent genetic resources for the breeding of new com varieties, it is still necessary to further explore more yield-related trait genes and analyze their utilization pathways.

The information disclosed in this Background section is merely intended to enhance an understanding of the general background of the present application and should not be construed as an admission or in any way implying that the information constitutes related art that is already known to those skilled in the art.

### SUMMARY

Based on 1×9801 and the Chang7-2 near isogenic line of 1×9801, the inventor cloned the gene KWE2 that controls corn ear grain weight and yield (kernel row number and kernel number per row are collaboratively determined), and provided a way to utilize it. The molecular marker InDel1 was developed: the KWE2 gene and its rice homologous gene were knocked out by CRISPR/Cas9, and the knockout mutant (KN5585 background) and Mu insertion mutant (W22 background) were used to cross with conventional inbred lines to clarify the yield-increasing effect of the KWE2 gene on hybrids. Based on the homologous genes of corn KWE2 in rice, tobacco, and Arabidopsis, the corresponding knockout mutations were created and its performance on the ear-grain weight trait and its effect on yield improvement were clarified. The present application provides technical support for using the KWE2 gene to improve the yield of various crops.

One of the purposes of the present application is to provide a new DNA sequence and cDNA sequence of the gene KWE2 that regulates corn ear grain weight and yield, and the amino acid sequence of the functional protein encoded by the gene KWE2. The gene encodes a SKP1-interacting partner 15 protein, and the down-regulated expression or knockout of this gene increases ear grain weight and yield.

A first aspect of the present application relates to a DNA sequence of the gene KWE2 for regulating the corn ear weight and yield, which is an isolated polynucleotide sequence, including a nucleotide sequence selected from the following group consisting of:
(1) a DNA molecule shown in SEQ ID NO. 1 or SEQ ID NO. 2 (cloned from the genomic DNA of the corn inbred lines Chang 7-2 and 1×9801 respectively);
(2) a cDNA molecule shown in SEQ ID NO.3 (cloned from the corn inbred line B73);
(3) a DNA molecule that can affect the phenotype of yield-related traits, formed through substitutions and/or insertions and/or deletions of one to several base and insertion/deletion/shift/inversion of large fragments nucleotide sequence, on the basis of SEQ ID NO.1. SEQ ID NO.2 or SEQ ID NO.3;
(4) a nucleotide sequence that can hybridize with the nucleotide sequence of SEQ ID NO.1. SEQ ID NO.2 or SEQ ID NO.3 under moderately stringent hybridization conditions and affect yield-related traits; and
(5) a nucleotide sequence that has more than 80% homology with the nucleotide sequence of SEQ ID NO.1, SEQ ID NO.2 or SEQ ID NO.3 and affects yield-related traits.

A second aspect of the present application aims to provide a protein encoded by the above-mentioned gene KWE2 for regulating corn ear grain weight and yield, including an amino acid sequence selected from the following group consisting of:
(1) a protein consisting of the amino acid sequence encoded by SEQ ID NO.1, SEQ ID NO.2 or SEQ ID NO.3, as shown in SEQ ID NO.4;
(2) a protein with SEQ ID NO.4 that has undergone substitution, deletion and/or addition of one or several amino acid residues and has affect yield-related traits;
(3) An amino acid sequence that has more than 70% homology with the amino acid sequence of SEQ ID NO. 4 and affects yield-related traits.

A third aspect of the present application relates to a promoter sequence for the expression of the above-mentioned gene KWE2 for regulating corn ear grain weight and yield, which includes a nucleotide sequence selected from the following group consisting of:
(1) the promoter sequence is the DNA sequence shown in SEQ ID NO.5, SEQ ID NO.6 or SEQ ID NO.7, which has the function of specifically initiating the transcription of the gene sequence shown in SEQ ID NO.1 and SEQ ID NO.2 or SEQ ID NO.3 in plants. SEQ ID NO.5 is the promoter sequence in the Chang7-2 donor in the 1×9801 background, and SEQ ID NO.6 is the promoter sequence in 1×9801, SEQ ID NO.7 is the promoter sequence in B73. Compared with SEQ ID NO.5. SEQ ID NO.6 lacks a 209 bp fragment (including a 103 bp PIF_Harbinger_TIR_transposon). Compared with SEQ ID NO.5, SEQ ID NO.7 inserts a 1596 bp fragment (containing an 846 bp helitron and a 407 bp helitron).
(2) the nucleotide sequence that can affect the transcription level of SEQ ID NO.1, SEQ ID NO.2 or SEQ ID NO.3, formed by through substitutions and/or insertions and/or deletions of one to several base as well as insertion/deletion/shift/inversion of large fragments of nucleotide sequences, on the basis of SEQ ID NO.5, SEQ ID NO.6 or SEQ ID NO.7.

A fourth aspect of the present application relates to a recombinant expression vector, an expression cassette, a transgenic cell line or a recombinant bacteria containing the gene and/or promoter.

A fifth aspect of the present application relates to a method and effect of providing the recombinant expression vector, the expression cassette, the transgenic cell line or the recombinant bacteria of the above gene and/or promoter for improving corn, rice, wheat and other plants. Plants that can be transformed in the present application can be monocotyledonous and dicotyledonous plants, including but not limited to corn, wheat, barley, rye, rice, rapeseed cotton, sweet potato, sunflower, potato, soybean, pea, endive, lettuce, cabbage, cauliflower, onion, garlic, spinach, Chinese cabbage, pak choi, cabbage, radish, pumpkin, apple, pear, strawberry, pineapple, tomato, sorghum, carrot, eggplant, cucumber, zucchini, poplar, paper towel, paulownia, switchgrass, alfalfa, peony, Chinese rose, rose, chrysanthemum, Arabidopsis, etc.

A sixth aspect of the present application relates to a method for improving plant traits using the KWE2 gene. The method includes preparing the plant containing the recombinant expression vector, the expression cassette, the transgenic cell line or the recombinant bacteria of the gene and/or promoter of the present application, or appropriately adjusting the expression level of the KWE2 gene in the plant, or appropriately changing biological activity of the KWE2 protein, or crossing the plant containing the KWE2 gene that regulates corn ear grain weight and yield according to the present application with another plant. The traits include improved yield and enhanced hybrid vigor in plants that produce or knock out the KWE2 gene that regulates com ear weight and yield.

One or more technical solutions in the embodiments of the present application have at least any of the following technical effects or advantages.

The KWE2 gene, which regulates corn ear grain weight and yield, is cloned, the functional nucleotide sequence that causes the differential expression of the KWE2 gene is identified, the functional molecular marker InDel1 is developed and the use is provided. On the other hand, the KWE2 gene is knocked out by CRISPR/Cas9, and the knockout mutant (KN5585 background) and Mu insertion mutant (W22 background) are used to cross with conventional inbred lines to clarify the effect of KWE2 gene on hybrid vigor performance of traits such as ear grain weight, 100-grain weight, yield, and plant height. Based on the homologous genes of the gene KWE2 in corn, rice and Arabidopsis, corresponding knockout mutations is created to clarify its enhance effect on yield improvement and hybrid vigor performance in multiple important agronomic traits, which provides technical support for using KWE2 gene to improve the yield of various crops and regulate hybrid vigor.

Utilizing the gene and method of the present application can facilitate the creation of high-yield breeding materials for crops such as corn, rice, and wheat, shorten the breeding cycle of new crop varieties, reduce breeding costs, and improve breeding efficiency.

### BRIEF DESCRIPTION OF THE DRAWINGS

FIG. 1 is a schematic structural diagram of a KWE2 gene according to an embodiment of the present application.

FIG. 2 is a construction diagram of a KWE2 gene CRISPR-Cas9 knockout vector according to an embodiment of the present application.

FIG. 3 is a schematic diagram of the editing of a corn knockout mutant KWE2 gene according to an embodiment of the present application.

FIG. 4 is a distribution diagram of InDel1 in inbred lines according to an embodiment of the present application: lanes 1-17 respectively represent inbred ensemble 3 (Zong3), Qi319, Yudan 888 male parent (T4691), Yudan 888 female parent (15S717), Dan340, B73, Zheng58, Chang7-2, PH4CV, PH6WC, C8605, U8112, 7884-4Ht, 1×9801, 1×9801^{KWE2}, Jing724, and Shen5003.

FIG. 5 is a comparison chart of the grain weight and yield per ear of the test-cross progeny of the corn knockout mutant according to an embodiment of the present application, bar=5 cm.

FIG. 6 is a phenotypic diagram of the test-cross progeny of the corn knockout mutant according to an embodiment of the present application, bar=20 cm.

FIG. 7 is a comparison diagram of the grain weight and yield per ear of the test-cross progeny of the com Mu mutant according to an embodiment of the present application, bar=5 cm.

FIG. 8 is a phenotypic diagram of the test-cross progeny of the corn Mu mutant according to an embodiment of the present application, bar=20 cm.

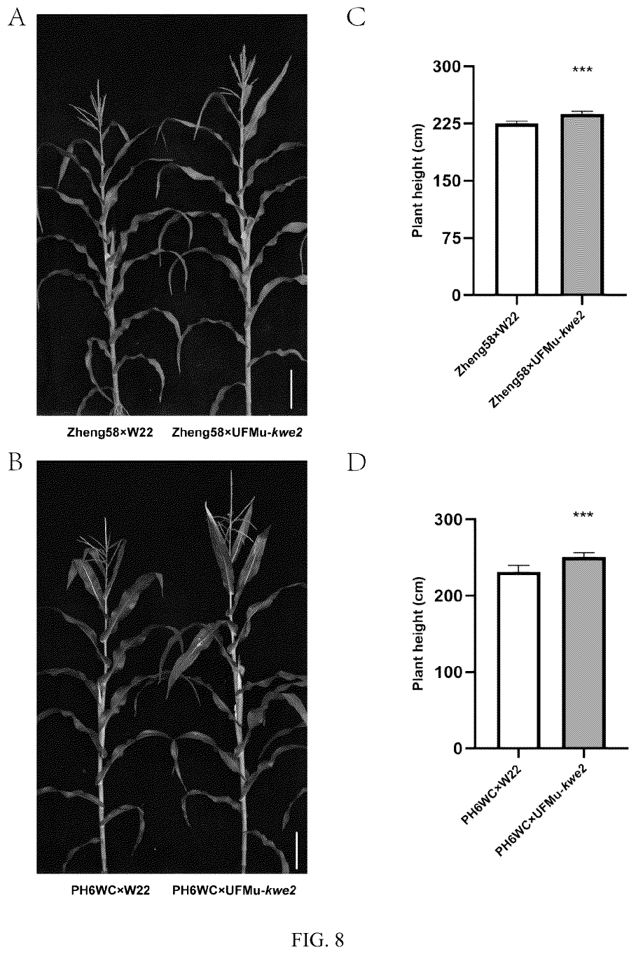

FIG. 9 is a diagram of a rice CRISPR-Cas9 knockout vector and overexpression vector according to an embodiment of the present application.

FIG. 10 is a diagram of the panicle phenotype of the knockout and overexpression lines of the rice KWE2 homologous gene according to an embodiment of the present application, bar=5 cm.

FIG. 11 is a phenotypic diagram of the EMS strain of the Arabidopsis thaliana KWE2 homologous gene according to an embodiment of the present application.

### DETAILED DESCRIPTION OF THE EMBODIMENTS

Definitions and explanations of related terms:

The term “ear grain weight and yield regulatory gene” refers to a nucleotide sequence with the ability to encode a protein, which specifically encodes a protein active polypeptide with the function of regulating ear-grain weight and yield, such as NO. 803-2032 nucleotide sequence of SEQ ID NO. 1 and its degenerate sequence, NO. 733-1962 nucleotide sequence of SEQ ID NO. 2 and its degenerate sequence, and NO. 203-1432 nucleotide sequence of SEQ ID NO. 3 and its degenerate sequence.

The term “degenerate sequence” refers to a sequence in which one or more codons in the nucleotide sequence located in the coding frame of SEQ ID NOs. 1-3 are replaced by degenerate codons encoding the same amino acid. Due to the degeneracy of codons, most degenerate sequences with as low as 70% homology to the coding frame nucleotide sequence of SEQ ID NOs. 1-3 can also encode amino acid sequence encoded by SEQ ID NOs. 1-3.

The “ear grain weight and yield regulatory genes” also include nucleotide sequences that can hybridize with the nucleotide sequences of SEQ ID NOs. 1-3 under moderately stringent conditions or under more highly stringent conditions. Moderately stringent conditions can include hybridization and membrane washing at 65° C. in a solution of 0.1×SSPE (or 0.1×SSC) and 0.1% (w/v) SDS.

The “genes that regulate ear grain weight and yield” also include at least 70% homology with the nucleotide sequences of SEQ ID NOs. 1-3, preferably 80%, 82%, 85%, 86%, 88%, 89% homology, more preferably 90%, 91%, 92%, 93%, 94% homology, the best has at least 95%, 96%, 97%, 98%, 99% homology to nucleotide sequences.

The “gene for regulating ear grain weight and yield” also include variant forms of the SEQ ID NOs. 1-3 open reading frame sequences that encode proteins with the same function as the natural gene KWE2 for regulating ear grain weight and yield. These variant forms include but are not limited to: deletion, insertion and/or substitution of one or several nucleotides, and addition of several nucleotides (usually within 60, preferably within 30, more preferably within 10, the best is within 5) at 5′ or 3′ end.

The “gene for regulating ear grain weight and yield” also includes the ability to translate a type of amino acid sequence that has the function of regulating corn ear grain weight and yield, such as the amino acid sequence of SEQ ID NO. 4. This type of amino acid sequence also includes a variant form of SEQ ID NO. 4 that has the same function of naturally regulating com ear grain weight protein. These variant forms include but are not limited to: deletion, insertion and/or substitution of one or several amino acids, and addition of one or several (usually within 20, preferably within 10, more preferably within 5) amino acids at the C-terminal and/or N-terminal. In this field, substitution with amino acids with same or similar properties usually does not change the function of the protein: adding one or several amino acids at the C-terminal and/or N-terminal usually does not change the function of the protein.

The “gene for regulating ear grain weight and yield” also includes the nucleotide sequence that can regulate the expression levels of corn ear grain weight and yield genes, such as the nucleotide sequences of SEQ ID NOs. 5-7. This type of nucleotide sequence also includes variant forms of SEQ ID NOs. 1-3 that can affect the expression level of ear grain weight and yield gene KWE2. These variant forms include, but are not limited to: substitutions and/or insertions and/or deletions of one to several base, as well as insertions/deletions/shifts/inversions of large fragments of nucleotide sequence.

In addition, the full-length nucleotide sequence of the “gene for regulating ear grain weight and yield” or its fragment can usually be obtained by PCR amplification, recombination or artificial synthesis. For the PCR amplification method, corresponding primers can be designed based on the relevant nucleotide sequences disclosed in this example, especially the open reading frame sequence, and can be amplified to relevant sequences using commercially available cDNA library or cDNA library prepared according to conventional methods known to those skilled in the art as a template. When the sequence is long, two or more nested PCR amplifications are usually required, and then the PCR amplification products are spliced together in the correct order. Once the relevant sequences are obtained, recombination can be used to obtain the relevant sequences in large quantities. It is usually cloned into a vector, and then the relevant sequence is isolated from the proliferating host cells through conventional methods such as cell transformation. In addition, mutations can also be introduced into the protein sequences in the embodiment through chemical synthesis. In addition to recombination, fragments of the proteins in the embodiment can also be produced by direct synthesis of polypeptides using solid phase techniques. Protein synthesis in vitro can be done manually or automatically. Each fragment of the protein in the embodiment can be chemically synthesized separately and then connected using chemical methods to produce a full-length protein molecule.

Particularly preferred is at least one protein of the gene KWE2 for regulating the corn ear grain weight and yield disclosed in the present application expressed in a higher plant. Once the desired nucleotide sequence is transformed into a specific plant species, it can be propagated in that species or transferred into other varieties of the same species (especially including commercial varieties) using conventional breeding techniques. The nucleotide sequence of each gene KWE2 for regulating corn ear grain weight and yield disclosed in the present application can be inserted into an expression cassette, or included in a non-pathogenic self-replicating virus, and then preferably, the expression cassette is stably integrated into the plant genome. Plants transformed in the present application can be monocotyledonous and dicotyledonous plants, including but not limited to com, wheat, barley, rye, rice, rapeseed, cotton, sweet potato, sunflower, potato, bean, pea, chicory, lettuce, cabbage, cauliflower, onion, garlic, spinach, Chinese cabbage, Chinese cabbage, cabbage, radish, pumpkin, apple, pear, strawberry, pineapple, tomato, sorghum, carrot, eggplant, cucumber, zucchini, poplar, paper towel, paulownia, switchgrass, alfalfa, peony, rose, rose, chrysanthemum, Arabidopsis, etc. By expressing the nucleotide sequence disclosed in the present application in transgenic plants, the biosynthesis of functional proteins capable of enhancing the corresponding heterosis performance is promoted in transgenic plants. In this way, transgenic plants with enhanced heterosis performance can be produced. In order to express the nucleotide sequences of the present invention in transgenic plants, the nucleotide sequences disclosed in the present application may need to be modified and optimized. All organisms have specific codon usage preferences, which are known in the art. It is possible to change the codons of the nucleotide sequence described in the present application to match plant preferences while maintaining the amino acids encoded by the nucleotide sequence. Moreover, encoding sequences with at least about 35% GC content. preferably more than about 45%, more preferably more than 50%, and most preferably more than about 60% GC content can best achieve high-level expression in plants. Furthermore, high levels of expression in plants are best achieved from coding sequences having a GC content of at least about 35%, preferably more than about 45%, more preferably more than 50%, and most preferably more than about 60%.

Some specific embodiments of the present application will be described in detail below: The following embodiments will facilitate a better understanding of the present application, but it should be understood that the scope of the present application is not limited by the specific embodiments. Based on the embodiments of the present application, all other embodiments obtained by those skilled in the art without any creative effort shall fall within the scope of the present application. The experimental methods described in the embodiments of the present application, unless otherwise specified, are all routine methods. Those skilled in the art should understand that, unless otherwise specified, the reagents, enzymes, etc. used in the following examples are all analytical pure grade reagents or enzymes that can be purchased from reagent companies. The materials, methods, and examples are illustrative only and are not intended to be limiting.

### Example 1. Cloning and Functional Verification of Gene KWE2

A major regulatory gene KWE2 that affects the ear grain weight and yield of hybrids was identified in 1×9801 and the Chang 7-2 near-isogenic line SSSL168 of 1×9801. The candidate gene was located in the 40 kb physical interval of corn chromosome 2 through map-based cloning, there are two protein-coding genes Zm00001eb076040 (Zm00001d002881) and Zm00001eb076050 (Zm00001d002882) in this interval in the B73 reference genome.

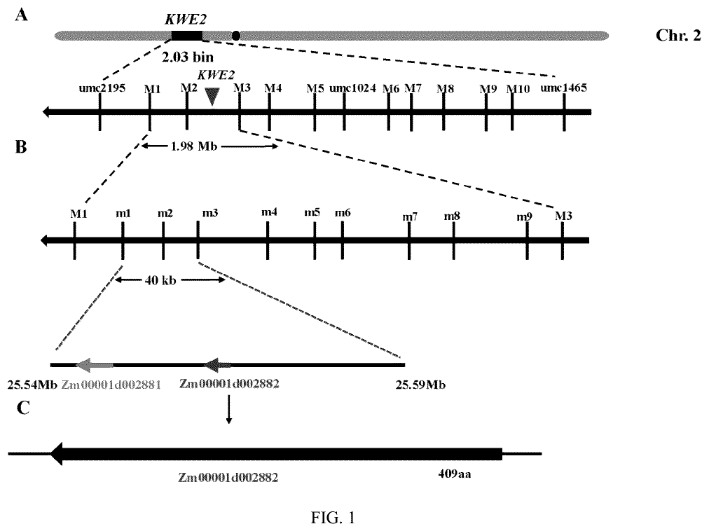

Sequencing analysis of the two candidate genes found that there were no amino acid sequence differences in the coding regions of the two genes. Only the promoter region of the Zm00001eb076050 gene had a long fragment insertion deletion. The promoter sequence in the Chang7-2 (Chang7-2) near-isogenic line (1×9801KWE2) of 1×9801 has a 209 bp fragment (including a 103 bp PIF_Harbinger_TIR_transposon) inserted compared to 1×9801. The promoter sequences in B73 and Zheng58 have a 1596 bp inserted fragment (including a 846 bp helitron and a 407 bp helitron) compared to 1×9801KWE2. Therefore, this gene was identified as a candidate gene related to ear grain weight and yield. Its functional annotation showed that Zm00001eb076050 encodes a SKP1-interacting partner 15 protein, with a full-length coding region of 1230 bp and no introns (see FIG. 1).

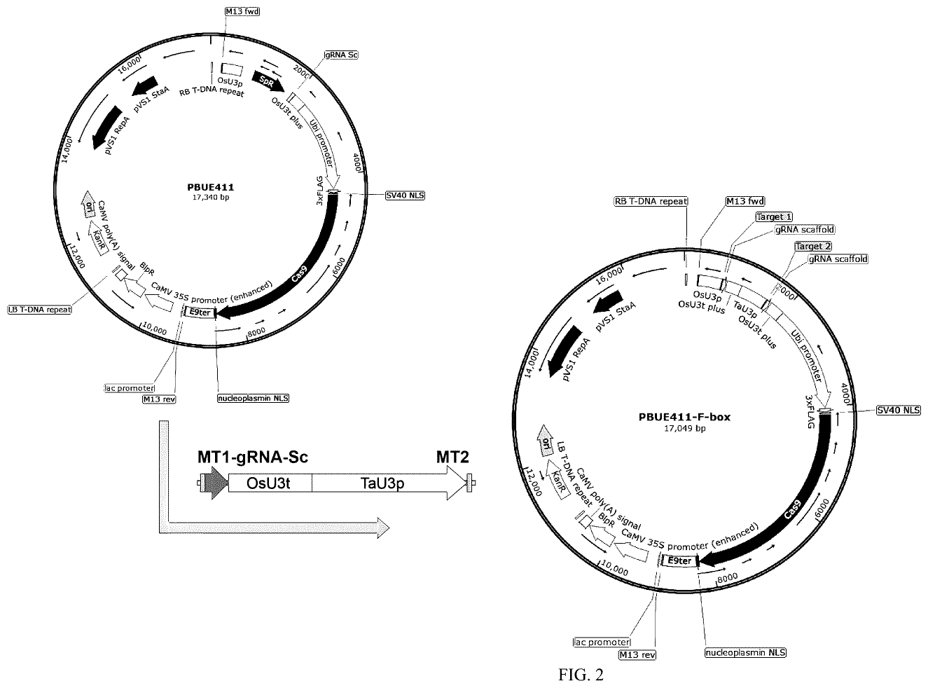

In order to verify the function of this gene, two target sites (Target1 and Targat2) were designed on the first exon of the KWE2 gene, and pCBC-MT1T2 was used as a template to amplify them using two primer pairs (MT1T2-F, MT1T2-F0, MT1T2-R0, MT1T2-R), connect the target site to the final vector pBUE411 to complete the construction of the CRISPR-Cas9 knockout vector (see FIG. 2), and the immature embryos of the corn inbred line KN5585 were transformed through Agrobacterium-mediated transformation.

Target1:
TCCTGCCCCGACCCGGCCG (CGG),
Target2:
CTTCTCCTGCCCGCTGGAC (AGG);
MT1T2-F:
AATAATGGTCTCAGGCGTCCTGCCCCGACCCGGCCG,
MT1T2-F0:
GTCCTGCCCCGACCCGGCCGGTTTTAGAGCTAGAAATAGC;
MT1T2-R0:
GTCCAGCGGGCAGGAGAAGCGCTTCTTGGTGCC,
MT1T2-R:
ATTATTGGTCTCTAAACGTCCAGCGGGCAGGAGAAG.

The CRISPR-Cas9 vector was transferred into Agrobacterium EHA105 by electroporation and identified by PCR. The freshly peeled young embryos of about 1 mm of corn inbred line KN5585 was used as material, and put into a 2 mL plastic centrifuge tube containing 1.8 mL of suspension. Approximately 150 immature embryos were processed within 30 minutes. The suspension was aspirated off, 1.0 mL of Agrobacterium suspension was added to the remaining corn embryos in the tube, and leave for 5 minutes. The immature embryos in the centrifuge tube was suspended and poured onto the co-culture medium. A pipette was used to remove excess Agrobacterium bacteria from the surface and co-culture for 3 days at 23° C. in the dark. After co-culture, the young embryos were transferred to resting medium, cultured in the dark at 28° C. for 6 days, and then placed on the selection medium containing 5 mg L-1 Bialaphos to select and culture for 2 weeks, then transferred to the selection medium containing 8 mg L-1 Bialaphos to select and culture for 2 weeks. The resistant calli were transferred to differentiation medium 1 and cultured at 25° C. under 5000 Lx light for 1 week. Then transfer the callus to differentiation medium 2 and culture it under light for 2 weeks: transfer the differentiated seedlings to rooting medium and culture them under 25° C. 5000 Lx light until they take root: transfer the seedlings to small pots for growth, transplant them to the greenhouse after a certain growth stage, and harvest the offspring seeds after 3-4 months.

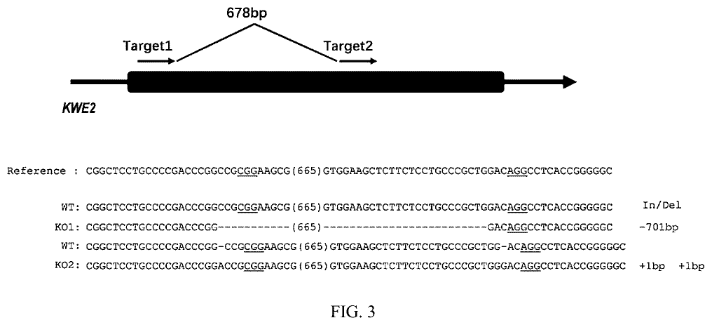

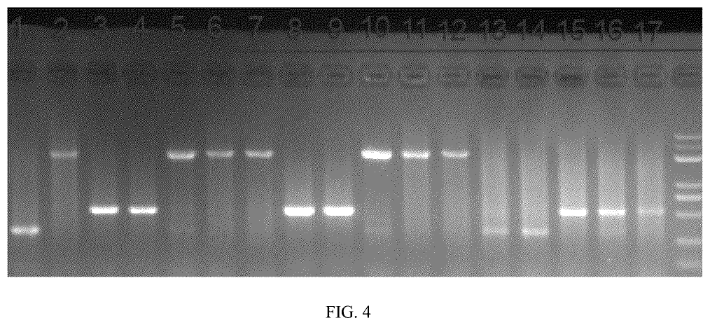

Knockout events were identified in the T2 generation (see FIG. 3), and homozygous knockout mutants were obtained. In order to clarify the yield-increasing effect of KWE2 gene in different genetic backgrounds, three homozygous knockout mutants were randomly selected and crossed with inbred lines Qi319, Zheng58, PH6WC, Shen5003, etc. of different heterotic groups. Difference analysis of KWE2 gene of inbred lines is shown in FIG. 4:
(1) First, the InDel1 marker primer pair is designed based on the 209 bp difference in the KWE2 gene promoter region of 1×9801^{KWE2} and 1×9801. Its sequence is as follows:

F:
5′-GTCAGCCCAGAACAAACG-3′,
R:
5′-GGTCCCACGAGAAGAACG-3′;

(2) Tissue samples from the inbred line to be identified is taken and genomic DNA is extracted;
(3) The resulting inbred line genomic DNA is used as a template and the InDel1 primer is used to perform PCR amplification;
(4) The length of the amplification product of the primer pair is detected: 515 bp corresponds to the 1×9801^{KWE2} genotype; 306 bp corresponds to the 1×9801 genotype; and 2111 bp corresponds to the B73 genotype. Inbred lines can be divided into three types through marker analysis. The banding patterns of 7884-4Ht are consistent with 1×801 and Zong3, the banding patterns of PH4CV, Jing724 and Shen5003 are consistent with 1×9801KWE2 and Chang7-2, and the banding patterns of Dan340, Qi319, PH6WC, C8605 and U8112 are consistent with Zheng58 and B73.

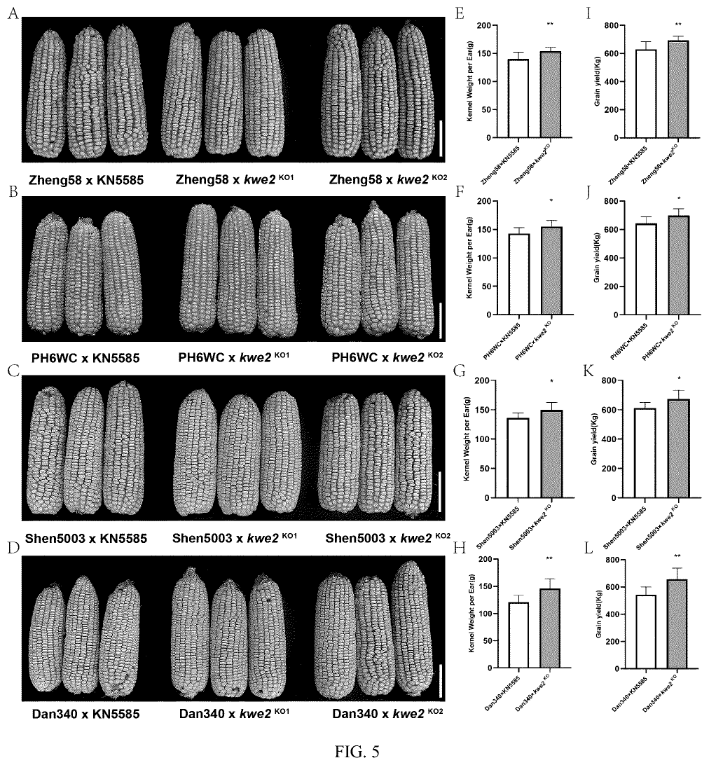

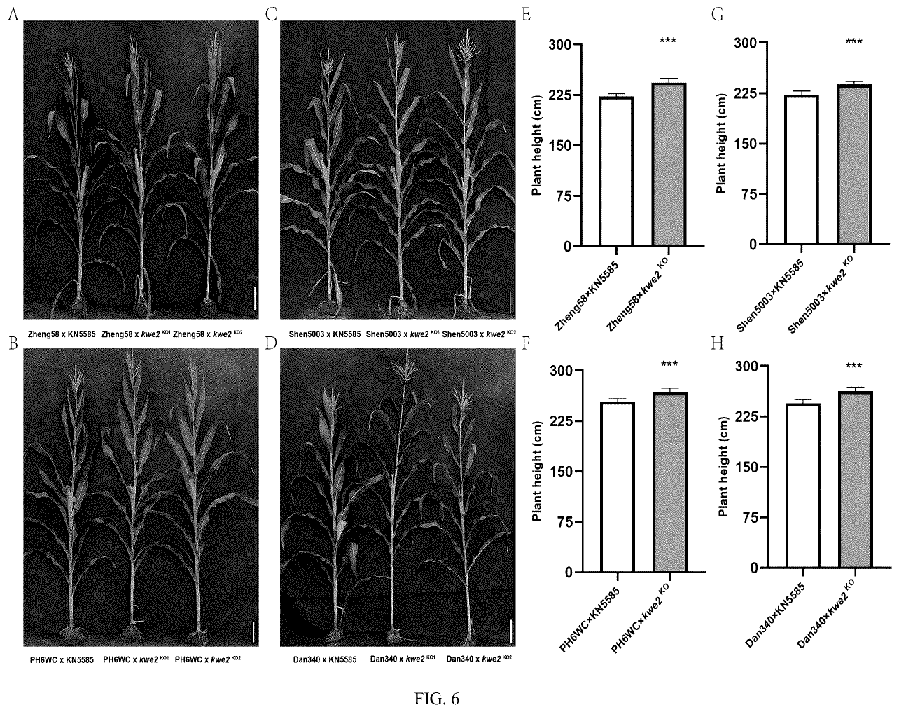

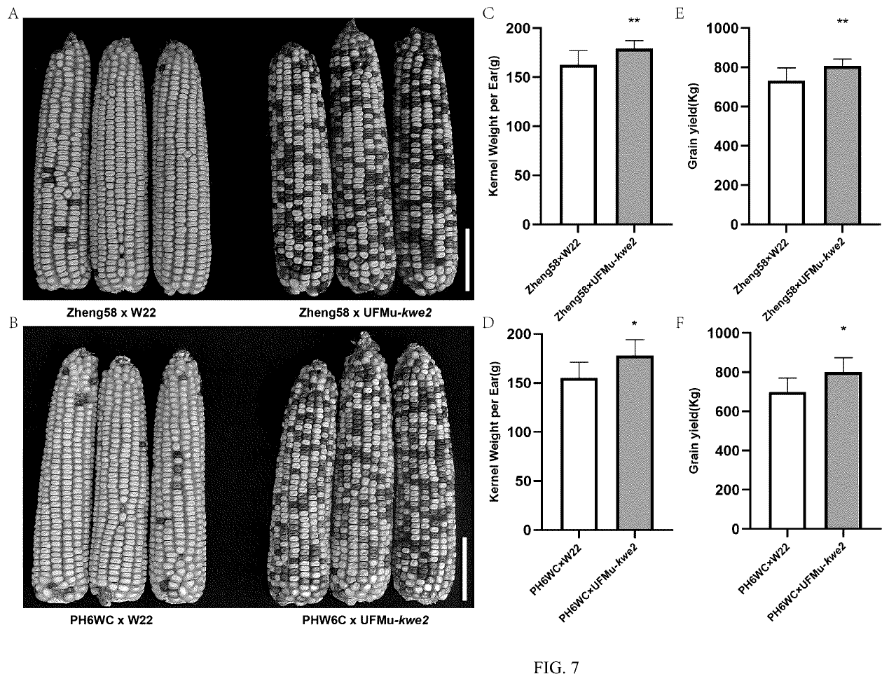

The above-mentioned hybrid combinations were planted at the transgenic base of the Henan Academy of Agricultural Sciences. At the same time, the hybrids of the transformation receptor KN5585 and these inbred lines were used as controls to conduct field identification of each hybrid combination. The identification results showed that the F1 hybrid between the knockout mutant and the inbred line had significantly higher traits such as ear grain weight and yield than the hybrid combination of KN5585 and the respective inbred lines (see FIG. 4 and FIG. 5). At the same time, the UFMu insertion mutant of the KWE2 gene (W22 background) in the American resource library was identified and purified and then crossed with the above-mentioned inbred lines, and its phenotypes were field identified in Hebi and Yuanyang in 2020. It was found that compared with the hybrid combination of W22 and the respective cross lines, the characteristics such as ear grain weight and yield were significantly increased (see FIGS. 6 and 7).

The above results indicate that the down-regulated expression or loss of function of KWE2 is beneficial to the improvement of corn ear weight and yield and the expression of heterosis for other major agronomic traits.

### Example 2: Analysis and Verification of the Role of KWE2 Gene in Other Species

To clarify whether homologous genes of KWE2 in other species have similar effects, CRISPR-Cas9 knockout of the homologous gene of KWE2 in rice was performed.

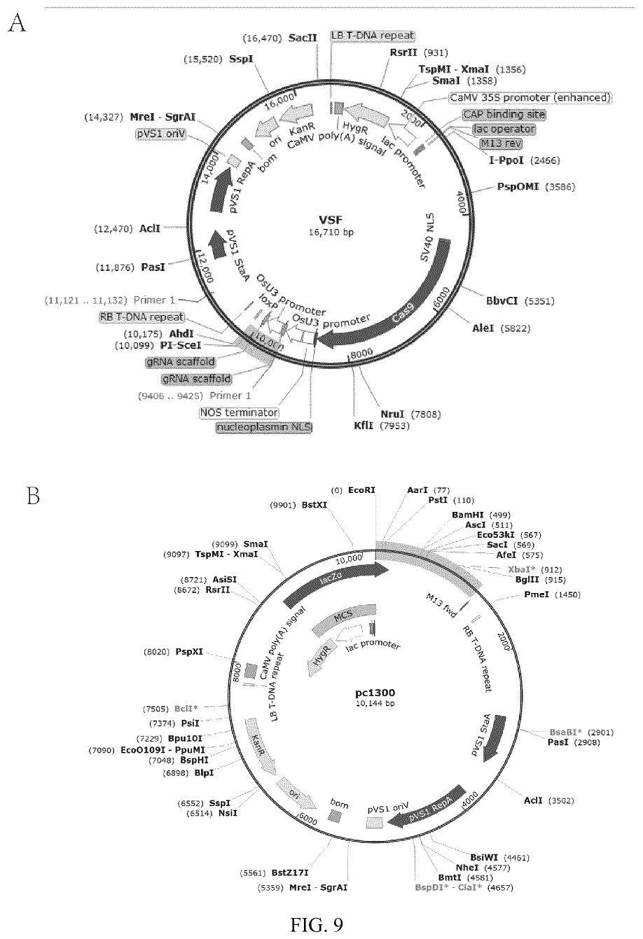

The specific operations are as follows:
1. Constructing the CRISPR-Cas9 vector of rice KWE2 gene cDNA (FIG. 9A), designing dual-target gRNA in the exon region (target 1: ccgataagctgeggcaatgccgg, target 2: aggacggaggaggcaaatcccgg), and using VSF-Y1 primer pair and VSF-B1 primer pair to construct intermediate vector. VSF-Y1 reaction system: 2 μL gRNA, 1.5 μL VSF-Y1 empty, 0.5 μL ECO31l endonuclease, 0.5 μL T4-DNA ligase, 1 μL T4 buffer, 4.5 μL H₂O, incubate at 37° C. for 2 h. VSF-B1 reaction system: 2 μL gRNA, 1.5 μL VSF-B1 empty, 0.5 μL ECO31l endonuclease, 0.5 μL T4-DNA ligase, 1 μL T4 buffer, 4.5 μL H₂O, incubate at 37° C. for 2 h. PCR reaction program: 95° C. for 10 min, 55° C. for 10 min, 14° C. for 5 min.

VSF--Y1 primer:
VSF--Y1 (+):
cagtGGTCTCaggcaccggcattgc cgcagcttat,
VSF--Y1 (−):
cagtGGTCTCaaaacataagctgcggcaatgccgg;
VSF--B1 primer:
VSF--B1 (+):
cagtGGTCTCaggcaggacggaggaggcaaatcc,
VSF--B1 (−):
cagtGGTCTCaaaacggatttgcctcctccgtcc.

2. Recombinant plasmid transformation
(1) Incubating 5 μL of ligation product with 200 μL of E. coli competent cells DH5a on ice for 30 min;
(2) Quickly placing it in a constant temperature water bath at 42° C. and heat shock conversion for 90 s;
(3) After 2 minutes of ice bath, adding 500 μL LB liquid culture medium and mix well;
(4) Shaking the bacteria at 200 rpm at 37° C. for 45 minutes to restore the cells to normal growth;
(5) Evenly spreading the bacterial solution on the LB solid culture medium plate;
(6) After 30 minutes, placing it in a 37° C. constant temperature incubator for overnight culture.

3. Selection of a single clone to extract plasmid. The specific steps are as follows:
(1) Picking a single clone from the LB solid medium plate and inoculating it into kanamycin-resistant LB liquid medium with a final concentration of 50 μg/mL, and culturing it at 37° C. overnight;
(2) Taking 4 mL of activated bacterial solution, centrifuge at 10,000 rpm for 2 minutes at room temperature, and discard the supernatant completely;
(3) Taking 250 μL of Solution I reagent containing RNase A and resuspend the bacterial clumps thoroughly;
(4) Taking 250 μL Solution II reagent to lyse the bacterial mass, and gently inverting it several times until the bacterial cells are transparent;
(5) Taking 350 μL Solution III reagent and inverting it several times until a white, compact floc is formed;
(6) Centrifuging at 12,000 rpm for 10 minutes at room temperature and taking the supernatant;
(7) Taking out the nucleic acid purification column from the kit and placing it on the collection tube;
(8) Transferring the clarified supernatant from step 6 above to the nucleic acid purification column, centrifuging at 12,000 rpm at room temperature for 1 min, and discard the filtrate;
(9) Adding 500 μL of Buffer W1 to the nucleic acid purification column, centrifuging at 12,000 rpm for 30 s at room temperature, and discarding the filtrate;
(10) Adding 700 μL of Buffer W2 to the nucleic acid purification column, centrifuging at 12,000 rpm for 30 s at room temperature, and discard the filtrate;
(11) Repeating the above steps (10);
(12) Placing the nucleic acid purification column on the collection tube and leaving it at room temperature at 12,000 rpm for 2 minutes to remove as much residual liquid as possible;
(13) Discarding the collection tube, place the nucleic acid purification column in a 1.5 mL EP tube, and adding 50 μL of eluent to elute the DNA attached to the nucleic acid purification column membrane (the eluent can be preheated in a constant temperature water bath at 65° C. to help elute DNA), let stand at room temperature for 2 minutes;
(14) Centrifuging at 12,000 rpm for 2 minutes at room temperature to elute the DNA attached to the nucleic acid purification column membrane, and storing it in a refrigerator at a low temperature of −40° C.;
(15) Taking a trace amount of the recovered product and use agarose gel electrophoresis with a concentration of 1% to detect the quality of the plasmid extraction.
(16) performing single clone sequencing on the plasmid.

4. Dual target enzyme digestion ligation system: 1 μL VSF-Y1-1 (plasmid), 1.5 μL VSF-B1-1 (plasmid), 0.5 μL LguI, 0.5 μL T4 ligase, 1 μL T4 buffer, 5.5 μL H₂O, after incubating in an incubator at 37° C. for 2 hours, transforming into E. coli, and performing bacterial detection after shaking and culturing.

5. Bacteria detection: 10 μL 2× Mix, 1 μL VSF-Y1+ (forward detection primer), 1 μL GET-(reverse detection primer), 8 μL H₂O for bacterial liquid detection, and the target band is 750 bp. Picking the correct single clone and inoculate the bacteria, extracting and sequencing the plasmid.

Reverse detection primer GET-:
ATACGAAGTTATGACTGCGACCGA.

6. Taking mature rice seeds, mechanically dehull them, select plump, plaque-free and high-quality seeds, disinfect them and inoculate them onto the corresponding culture medium to induce callus.

7. Picking a single colony of Agrobacterium, placing it in the culture medium, and culturing it with shaking.

8. Agrobacterium and callus co-culture
\textcircled{1} Putting the cultured bacterial liquid into a centrifuge tube, centrifuge to get the supernatant, and prepare Agrobacterium suspension:
\textcircled{2} Picking out the callus that has grown to a certain size and infect it with Agrobacterium suspension:
\textcircled{3} Placing the callus on the co-culture medium.

9. Filter
\textcircled{1} Taking out the callus:
\textcircled{2} Transferring the dried callus to the screening medium for the first screening:
\textcircled{3} Transducing the initial callus with resistant calli into new culture medium for the second screening.

10. Induction, differentiation and rooting of resistant callus
\textcircled{1} Picking the resistant callus, move it into a petri dish containing differentiation medium, seal it with a sealing film, and place it in a constant-temperature culture room to wait for differentiation into seedlings.
\textcircled{2} When the seedlings grow to about 1 cm, moving them to the rooting medium to strengthen the seedlings.

11. Extracting plant genomes by CTAB method, and detect positive plants by PCR.

At the same time, the overexpression vector of the rice KWE2 gene (FIG. 9B) was genetically transformed:
\textcircled{1} Taking 1 μL of overexpression vector plasmid and add 50 μL of EHA105 Agrobacterium competent cells;
\textcircled{2} After thoroughly mixing taking the mixture into an electroporation cup for electroporation. After electroporation, adding 1 mL of LB liquid culture medium. Mix thoroughly and transfer to a 1.5 mL Ep tube. Shake and incubate on a shaker at 30° C. and 180 rpm for 30 minutes. Take 50 μL of the activated Agrobacterium bacteria liquid and inoculate it on LB solid medium, and cultivate it in the dark at 30° C. for 48 hours.

\textcircled{3} Agrobacterium detection primer pair hyg (280)+:

5′ACGGTGTCGTCCATCACAGTTTGCC3′;
5′TTCCGGAAGTGCTTGACATTGGGGA3′.

hyg (280)−:

\textcircled{4} Selecting Minghui 86 rice grains with no mildew spots and normal bud openings,

disinfect with 75% alcohol for 1 minute, rinse with sterile water, 1 minute/time; disinfect with sodium hypochlorite for 20 minutes, rinse with sterile water 3 times, 1 minute/time: the resulting rice grains were inoculated into the induction medium and cultured under light at 26° C. for 20 days.

\textcircled{5} Picking Agrobacterium in the infection solution, prepare an Agrobacterium resuspension with OD600=0.2, pick the callus in an Erlenmeyer flask, add the Agrobacterium resuspension, and discard the bacterial solution after 10-15 minutes of infection. The calli were inoculated into co-culture medium and co-cultured at 20° C. for 48-72 h.

\textcircled{6} Inoculating the callus into the screening medium and cultivate it in the dark at 26° C. for 20-30 days: pick the positive callus to the second screening medium. During the callus picking process, be sure to select monoclonal callus and cultivate it in the dark at 26° C. for 7-10 days.

\textcircled{7} Inoculating the positive callus into the differentiation medium, and culture it in the light at 25-27° C. for 15-20 days. After differentiation of 2-5 cm buds, inoculate it into the rooting medium, and culture it in the light at 30° C. for 7-10 days.

\textcircled{8} Using CTAB method to extract rice genomic DNA, conduct PCR detection, and detect positive seedlings.

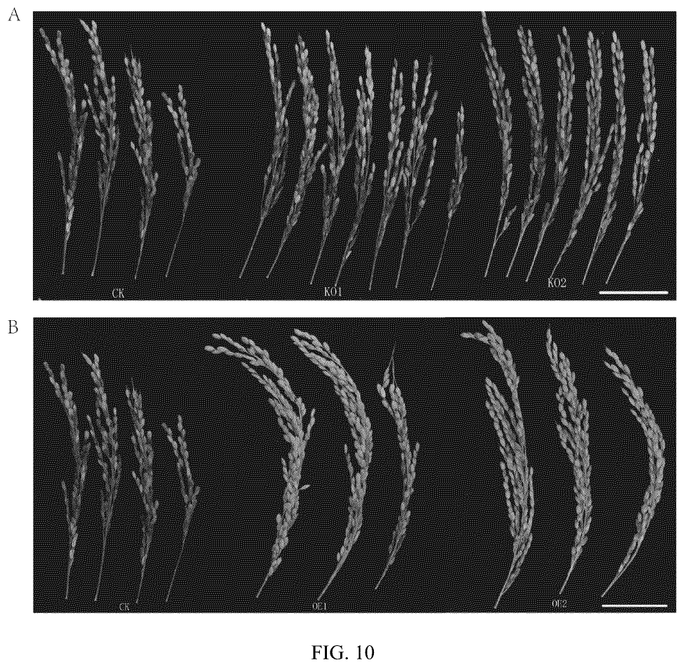

The T2 generation of transgenic positive plants was genotyped and homozygous CRISPR-Cas9 mutants and overexpression lines were obtained (FIG. 10). CRISPR-Cas9 knockout of KWE2 can increase the number of tillers and spikelets, while its overexpression reduces tillers and increases the number of spikelets.

It can be seen that the rice KWE2 gene negatively regulates the number of tillers and spikelets, but promotes the increase of ear grain number, thereby affecting the superior performance of hybrid yield.

### Example 3: Analysis and Verification of the Role of KWE2 Gene in Dicotyledonous Plants

To further clarify the role of KWE2 in dicots, a T-DNA insertion mutant (N666804/SALK_095140C, http://signal.salk.edu/cgi-bin/tdnaexpress) of the Arabidopsis KWE2 homologous gene (At1g76920) was performed phenotypic analysis. T-DNA is inserted into the 5′UTR (Untranslated region) of At1g76920, and the detection primer pair is

LP-5′-AGAACAGTACCATGGCGTGAC-3′
and
RP-5′-ACGTTCTGTCAAATCTGTCCC-3′.

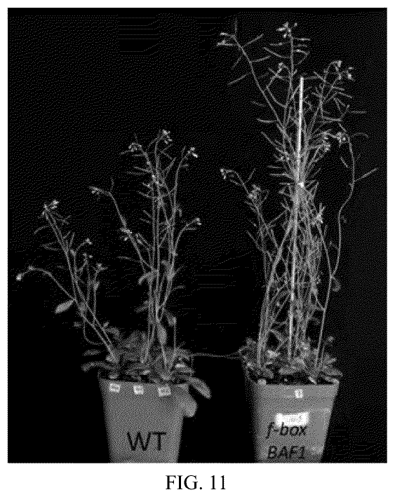

As can be seen from FIG. 11, the plant height, pod length and other traits of the KWE2 T-DNA insertion mutant are significantly better than the wild-type control, indicating that the KWE2 gene also has a role in regulating yield in dicotyledonous plants.

What is claimed is:

1. A gene KWE2 for regulating ear grain weight and yield, comprising a nucleotide sequence selected from at least one of the following:
a DNA molecule shown in SEQ ID NO.2; and
a DNA molecule, wherein in the DNA molecule, based on sequence shown in SEQ ID NO.1 or SEQ ID NO.2, through insertion and/or deletion of one to several bases, the gene is down-regulated or silenced to positively regulate the ear grain weight and the yield.

2. A protein encoded by the gene KWE2 for regulating the ear grain weight and yield of claim 1.

3. A promoter, used to regulate expression of the gene KWE2 for regulating the ear grain weight and yield of claim 1, wherein a nucleotide sequence of the promoter is shown in SEQ ID NO.5 or SEQ ID NO.6.

4. A recombinant expression vector, expression cassette, transgenic cell line or recombinant bacteria, comprising the gene KWE2 for regulating the ear grain weight and yield of claim 1 or/and the promoter of claim 3.

5. An InDel1 marker of a gene KWE2 for regulating ear grain weight and yield, wherein a fragment at a chr2: 25587558-25585754 position is deleted/inserted in a promoter region of a Zm000010002882 gene based on a fourth edition of corn reference genome B73.

6. A primer pair based on the InDel1 mark of claim 5, wherein a sequence of the primer pair is as follows:
F:
5′-GTCAGCCCAGAACAAACG-3′,
R:
5′-GGTCCCACGAGAAGAACG-3′.

7. A use of the gene KWE2 of claim 1, the promoter of claim 3, and the InDel1 marker of claim 5 in plant trait improvement, yield control, or/and heterotic variety/strain breeding.

8. The use of claim 7, wherein a plant gene KWE2 is silenced/down-regulated.

9. The use of claim 8, comprising the following steps:
constructing a knockout vector or a silencing vector of the plant gene KWE2 and transforming a plant, cultivating and screening out a corresponding homozygous mutant, and crossbreeding with a corresponding inbred line.

10. A method for identifying a genotype with excellent com car grain weight and yield, comprising:
taking a corn tissue sample to be identified and extracting genomic DNA;
using the genomic DNA as a template, and performing PCR amplification using a InDel1 primer of claim 6; and
detecting a length of a amplification product of a primer pair, wherein 515 bp corresponds to 1×9801^{KWE2} genotype, 306 bp corresponds to 1×9801 genotype, and 2111 bp corresponds to B73 genotype.


SEQUENCE LISTING (7 sequences)

SEQ ID NO: 1 - DNA, 2314 bp, synthetic construct
  source 1..2314: mol_type = other DNA
tatttaacaaaccaaataaaaatatgagaagtgagaagaagatagatcacgtcattgctcaaaccaaacaaccgaacgaaacccgtcgatcagaacccgtgctacccataatgaaagcccatgggcctaaaggcctagcccaccgttcttctcgtgggacccacacggagaaccgctctgccgacaggtggggtgacccgagccggccaggaaccaacgcgccgcgccgcccatttgcggcgtgcataccttttatttatcggctacccaacatggcagacagctgggccctggcacgcattattgccccacttgccaggcgaagtgagctgcccaccgcgctcctccgcgtcgctttgcgattttctggctcgttcccaccgctgcaacccggaacttgacgcagtctctccatgacaacatggcccacaagcgtgggtcccctactcacggactgtacacatatgaggtagagcaataccgcgtacttacagcccactccttacttttttagtttctgtctagttttgatgaggaacggaaacggaagcgccgctcaccaaggcgtaatgtctacccttggcctccttctcactgacatccgggtctcccgcagcacccacatgggtagaggaaaaacagtgggcaggtcgttctattctacggaaacggaacccacagcatcgtaggcgaaactgacaccctgtcccccgccttccccgacctcctcttcttctcctcgccggcccccgctatggtctcatccgtgtgttccccacctaaccccaccccccaatccgccatgcccggcggctcctgccccgacccggccgcggaagcgcagctcccgccgccgatccaccacctgccgccggacgcgctccacaacgtgctgctccggctgcagctgcgcgacgccgtcgtgtgccgcctcgtctcgcgcctcttccacgagaccctctcgccgcagctcctcgcgctgctgcccaccctgcgcctcctcctcctccgccacccgcgccccgacggcggcggctgcctccacgccttcgaccccgcgcgccgcacctggctccgcctccccttcgctcacttcctcccctaccagtccttctcccccgtcgcctcatccacgtccctcctctacctctgggtcgagacctcctcctccacctcgcccctagccctccagtccaccactacgtcttcctccgccgcctcgcccgcgcacccgctcaaatcgctcgccgtctgcaaccccttcgccggaacgtaccgcctcctgccccagctcggatccgcctgggcgcgccacggcaccgtcctcgccggccccggcggcatggtgctcgtcctcaccgagctcgcagcgctgtcctacacaccctccgaatccggcaagtggatgaagcacccgctctcgctgccgtccaagccgcggagccccatactggcctccgccgccgtcttcgccctctgcgacgtcggcacgccgtggcgcagccagtggaagctcttctcctgcccgctggacaggctcaccgggggctgggcgcccgtggagcgagcagcgtggggagacgtcttcgaggttctcaagcgtcctcggctccttgccggtgccggcggccgccgggttcttatggtcggcgggctcagatcctcgttcgccatggacgcgccgtgctccaccgttctcatactccgtctagatctagccaccatggagtgggacgaagctgggcgcatgccgcccaatatgtaccgctgcttcactggcctctgcgaggctgcttcacagggcaacgccatgcctgccaccgctggcgggggtaacaataaggtcaaggttttcggaggcgatggaaaggtgtggttcgctggtaagcgagtgcgcgggaagcttgcaatgtgggaggaagatgagacggggagcagtggcaagtgggactgggtggatggtgttcctggttatgatgatggtgtgtatcgtggatttgtgttcgatagtggattcacagcgattccttgatgcacatggaaaacgtatcctagtctggtgcactcaaattgaggtttaatttgacccaggacatctatctatcgcatgcacaggatctgttatgcccacacattttatttacagtctttagcatggtgagaactctagttgttgtacttgattaactgaaaagatcagtaagctgtttgtttcagacaattaaatgctgtagatcatacttgtcagaaacctgatatgttgctttgatagtgtcagaatttttgccatttggtaaacattttctgcttggtc

SEQ ID NO: 2 - DNA, 2244 bp, synthetic construct
  source 1..2244: mol_type = other DNA
accgaacgaaacccgtcgatcagaacccgtgctacccataatgaaagcccatgggcctaaaggcctagcccaccgttcttctcgtgggacccacacggagaaccgctctgccgacaggtggggtgacccgagccggccaggaaccaacgcgccgcgccgcccatttgcggcgtgcataccttttatttatcggctacccaacatggcagacagctgggccctggcacgcattattgccccacttgccaggcgaagtgagctgcccaccgcgctcctccgcgtcgctttgcgattttctggctcgttcccaccgctgcaacccggaacttgacgcagtctctccatgacaacatggcccacaagcgtgggtcccctactcacggactgtacacatatgaggtagagcaataccgcgtacttacagcccactccttacttttttagtttctgtctagttttgatgaggaacggaaacggaagcgccgctcaccaaggcgtaatgtctacccttggcctccttctcactgacatccgggtctcccgcagcacccacatgggtagaggaaaaacagtgggcaggtcgttctattctacggaaacggaacccacagcatcgtaggcgaaactgacaccctgtcccccgccttccccgacctcctcttcttctcctcgccggcccccgctatggtctcatccgtgtgttccccacctaaccccaccccccaatccgccatgcccggcggctcctgccccgacccggccgcggaagcgcagctcccgccgccgatccaccacctgccgccggacgcgctccacaacgtgctgctccggctgcagctgcgcgacgccgtcgtgtgccgcctcgtctcgcgcctcttccacgagaccctctcgccgcagctcctcgcgctgctgcccaccctgcgcctcctcctcctccgccacccgcgccccgacggcggcggctgcctccacgccttcgaccccgcgcgccgcacctggctccgcctccccttcgctcacttcctcccctaccagtccttctcccccgtcgcctcatccacgtccctcctctacctctgggtcgagacctcctcctccacctcgcccctagccctccagtccaccactacgtcttcctccgccgcctcgcccgcgcacccgctcaaatcgctcgccgtctgcaaccccttcgccggaacgtaccgcctcctgccccagctcggatccgcctgggcgcgccacggcaccgtcctcgccggccccggcggcatggtgctcgtcctcaccgagctcgcagcgctgtcctacacaccctccgaatccggcaagtggatgaagcacccgctctcgctgccgtccaagccgcggagccccatactggcctccgccgccgtcttcgccctctgcgacgtcggcacgccgtggcgcagccagtggaagctcttctcctgcccgctggacaggctcaccgggggctgggcgcccgtggagcgagcagcgtggggagacgtcttcgaggttctcaagcgtcctcggctccttgccggtgccggcggccgccgggttcttatggtcggcgggctcagatcctcgttcgccatggacgcgccgtgctccaccgttctcatactccgtctagatctagccaccatggagtgggacgaagctgggcgcatgccgcccaatatgtaccgctgctttactggcctctgcgaggctgcttcacagggcaacgccatgcctgccaccgctggcgggggtaacaataaggtcaaggttttcggaggcgatggaaaggtgtggtttgctggtaagcgagtgcgcgggaagcttgcaatgtgggaggaagatgagacggggagcagtggcaagtgggactgggtggatggtgttcctggttatgatgatggtgtgtatcgtggatttgtgttcgatagtggattcacagcgattccttgatgcacatggaaaacgtatcctagtctggtgcactcaaattgaggtttaatttgacccaggacatctatctatcgcatgcacaggatctgttatgcccacacattttatttacagtctttagcatggtgagaactctagttgttgtacttgattaactgaaaagatcagtaagctgtttgtttcagacaattaaatgctgtagatcatacttgtcagaaacctgatatgttgctttgatagtgtcagaatttttgccatttggtaaacattttctgcttggtc

SEQ ID NO: 3 - DNA, 1625 bp, synthetic construct
  source 1..1625: mol_type = other DNA
tccgggtctcccgcagcacccacatgggtagaggaaaaacagtgggcaggtcgttctattctacggaaacggaacccacagcatcgtaggcgaaactgacaccctgtcccccgccttccccgacctcctcttcttctcctcgccggcccccgctatggtctcatccgtgtgttccccacctaaccccaccccccaatccgccatgcccggcggctcctgccccgacccggccgcggaagcgcagctcccgccgccgatccaccacctgccgccggacgcgctccacaacgtgctgctccggctgcagctgcgcgacgccgtcgtgtgccgcctcgtctcgcgcctcttccacgagaccctctcgccgcagctcctcgcgctgctgcccaccctgcgcctcctcctcctccgccacccgcgccccgacggcggcggctgcctccacgccttcgaccccgcgcgccgcacctggctccgcctccccttcgctcacttcctcccctaccagtccttctcccccgtcgcctcatccacgtccctcctctacctctgggtcgagacctcctcctccacctcgcccctagccctccagtccaccactacgtcttcctccgccgcctcgcccgcgcacccgctcaaatcgctcgccgtctgcaaccccttcgccggaacgtaccgcctcctgccccagctcggatccgcctgggcgcgccacggcaccgtcctcgccggccccggcggcatggtgctcgtcctcaccgagctcgcagcgctgtcctacacaccctccgaatccggcaagtggatgaagcacccgctctcgctgccgtccaagccgcggagccccatactggcctccgccgccgtcttcgccctctgcgacgtcggcacgccgtggcgcagccagtggaagctcttctcctgcccgctggacaggctcaccgggggctgggcgcccgtggagcgagcagcgtggggagacgtcttcgaggttctcaagcgtcctcggctccttgccggtgccggcggccgccgggttcttatggtcggcgggctcagatcctcgttcgccatggacgcgccgtgctccaccgttctcatactccgtctagatctagccaccatggagtgggacgaagctgggcgcatgccgcccaatatgtaccgctgcttcactggcctctgcgaggctgcttcacagggcaacgccatgcctgccaccgctggcgggggtaacaataaggtcaaggttttcggaggcgatggaaaggtgtggttcgctggtaagcgagtgcgcgggaagcttgcaatgtgggaggaagatgagacggggagcagtggcaagtgggactgggtggatggtgttcctggttatgatgatggtgtgtatcgtggatttgtgttcgatagtggattcacagcgattccttgatgcacatggaaaacgtatcctagtctggtgcactcaaattgaggtttaatttgacccaggacatctatctatcgcatgcacaggatctgttatgcccacacattttatttacagtctttagcatggtgagaactctagttgttgtacttgattaactgaaaagatcagtaagctgtttgtttcagacaattaa

SEQ ID NO: 4 - protein, 409 aa, synthetic construct
MPGGSCPDPAAEAQLPPPJHHLPPDALHNVLLRLQLRDAVVCRLVSRLFHERLSPQLLALLPRLRLLLLRHPRPDGGGCLHAFDPARRRWLRLPFAHFLPYQSFSPVASSRSLLYLWVERSSSRSPLALQSRRRSSSAASPAHPLKSLAVCNPFAGRYRLLPQLGSAWARHGRVLAGPGGMVLVLRELAALSYRPSESGKWMKHPLSLPSKPRSPJLASAAVFALCDVGRPWRSQWKLFSCPLDRLRGGWAPVERAAWGDVFEVLKRPRLLAGAGGRRVLMVGGLRSSFAMDAPCSRVLJLRLDLARMEWDEAGRMPPNMYRCFRGLCEAASQGNAMPARAGGGNNKVKVFGGDGKVWFAGKRVRGKLAMWEEDERGSSGKWDWVDGVPGYDDGVYRGFVFDSGFRAJP

SEQ ID NO: 5 - DNA, 2053 bp, synthetic construct
  source 1..2053: mol_type = other DNA
tttctgttctagtgtcgggttcattaatgttcactgtttattgttgtgtatactactctaggtcggttcattgttgtgttaattccatgtatgttgttgtcactactattcgttgctttcattgctgttatctatttatgcacacatgaattagattaagatgatgaggacatggaattttttttgaagagttatgaacaaattgacaatacatatgatgcttggtacgttacactacgacacatacatgaataaagaatagtatagaaatccagtcgagtccagtttccaatgggttatgacatccctaggtaatacgacagcttgattcaacatgtttaggatgcacaatgatgtgttccataaactgcataatgtactagtggagtcacatggtctgaaatcaactacaaaaatgtcaccaattgaggcactaggtcaatatttatggatgtgtggtgcccctcaaagtatgagacatgacgaaaatcgtttttgtagatcaacatatacgcgcagtaagaagtttagtgaagtattgttaagtgtcaataaacttgcagcagatattattaagccattagatcctgaatttagtctcaccattttagtctcatttagtccctaaattgccaaacagtgggactaaaatagagactaaacttttttagtctctagtctctaaagaggtgactaaaagagactaaactatataaattttaccttttgtctcttctttatttcagtaattttggtcctcctatggtttcatttaatgcgttttgaataattttagtctttagaaccaaacggagtagagactaaactgtagtattctaactaaactttaatccctaaactaaagaaatcaaacgggtccttagtcactaaactttagtcagcccagaacaaacggggcctaagtgtttacttgtgcttattcttcaaatagaaaagtaaaaaatcaccttgaaaaccaattgtacctagtgcagagaggtaaataaaacgacattgagagttaaaggacgaaatttgctcggtttcaaacttcgaatagaaaacttagatggcaattatatacaccagtttagaatccttcttttttattgtgtttggtttatggagtcacccaatcattttgatgcatcataagttcattccatcaattttggtggaatcaactcacttctcacgtatatactaattattagcttataagaaatgaagtggtgatggataaacttattctatttaacaaaccaaataaaaatatgagaagtgagaagaagatagatcacgtcattgctcaaaccaaacaaccgaacgaaacccgtcgatcagaacccgtgctacccataatgaaagcccatgggcctaaaggcctagcccaccgttcttctcgtgggacccacacggagaaccgctctgccgacaggtggggtgacccgagccggccaggaaccaacgcgccgcgccgcccatttgcggcgtgcataccttttatttatcggctacccaacatggcagacagctgggccctggcacgcattattgccccacttgccaggcgaagtgagctgcccaccgcgctcctccgcgtcgctttgcgattttctggctcgttcccaccgctgcaacccggaacttgacgcagtctctccatgacaacatggcccacaagcgtgggtcccctactcacggactgtacacatatgaggtagagcaataccgcgtacttacagcccactccttacttttttagtttctgtctagttttgatgaggaacggaaacggaagcgccgctcaccaaggcgtaatgtctacccttggcctccttctcactgacatccgggtctcccgcagcacccacatgggtagaggaaaaacagtgggcaggtcgttctattctacggaaacggaacccacagcatcgtaggcgaaactgacaccctgtcccccgccttccccgacctcctcttcttctcctcgccggcccccgctatggtctcatccgtgtgttccccacctaaccccaccccccaatccgcc

SEQ ID NO: 6 - DNA, 1844 bp, synthetic construct
  source 1..1844: mol_type = other DNA
tttctgttctagtgtcgggttcattaatgttcactgtttattgttgtgtatactactctaggtcggttcattgttgtgttaattccatgtatgttgttgtcactactattcgttgctttcattgctgttatctatttatgcacacatgaattagattaagatgatgaggacatggaattttttttgaagagttatgaacaaattgacaatacatatgatgcttggtacgttacactacgacacatacatgaataaagaatagtatagaaatccagtcgagtccagtttccaatgggttatgacatccctaggtaatacgacagcttgattcaacatgtttaggatgcacaatgatgtgttccataaactgcataatgtactagtggagtcacatggtctgaaatcaactacaaaaatgtcaccaattgaggcactaggtcaatatttatggatgtgtggtgcccctcaaagtatgagacatgacgaaaatcgtttttgtagatcaacatatacgcgcagtaagaagtttagtgaagtattgttaagtgtcaataaacttgcagcagatattattaagccattagatcctgaatttagtctcaccattttagtctcatttagtccctaaattgccaaacagtgggactaaaatagagactaaacttttttagtctctagtctctaaagaggtgactaaaagagactaaactatataaattttaccttttgtctcttctttatttcagtaattttggtcctcctatggtttcatttaatgcgttttgaataattttagtctttagaaccaaacggagtagagactaaactgtagtattctaactaaactttaatccctaaactaaagaaatcaaacgggtccttagtcactaaactttagtcagcccagaacaaacggggcctaagtgtttacttgtgcttattcttcaaatagaaaagtaaaaaatcaccttgaaaaccaattgtacctagtgcagagaggtaaataaaacgacattgagagttaaaggacgaaatttgctcggtttcaaacttcgaatagaaaacttagatggcaattatatacaccagtttagaatccttcttttttattgaccgaacgaaacccgtcgatcagaacccgtgctacccataatgaaagcccatgggcctaaaggcctagcccaccgttcttctcgtgggacccacacggagaaccgctctgccgacaggtggggtgacccgagccggccaggaaccaacgcgccgcgccgcccatttgcggcgtgcataccttttatttatcggctacccaacatggcagacagctgggccctggcacgcattattgccccacttgccaggcgaagtgagctgcccaccgcgctcctccgcgtcgctttgcgattttctggctcgttcccaccgctgcaacccggaacttgacgcagtctctccatgacaacatggcccacaagcgtgggtcccctactcacggactgtacacatatgaggtagagcaataccgcgtacttacagcccactccttacttttttagtttctgtctagttttgatgaggaacggaaacggaagcgccgctcaccaaggcgtaatgtctacccttggcctccttctcactgacatccgggtctcccgcagcacccacatgggtagaggaaaaacagtgggcaggtcgttctattctacggaaacggaacccacagcatcgtaggcgaaactgacaccctgtcccccgccttccccgacctcctcttcttctcctcgccggcccccgctatggtctcatccgtgtgttccccacctaaccccaccccccaatccgcc

SEQ ID NO: 7 - DNA, 3649 bp, synthetic construct
  source 1..3649: mol_type = other DNA
tttctgttctagtgtcgggttcattaatgttcactgtttattgttgtgtatactactctaggtcggttcattgttgtgttaattccatgtatgttgttgtcactactattcgttgctttcattgctgttatctatttatgcacacatgaattagattaagatgatgaggacatggaattttttttgaagagttatgaacaaattgacaatacatatgatgcttggtacgttacactacgacacatacatgaataaagaatagtatagaaatccagtcgagtccagtttccaatgggttatgacatccctaggtaatacgacagcttgattcaacatgtttaggatgcacaatgatgtgttccataaactgcataatgtactagtggagtcacatggtctgaaatcaactacaaaaatgtcaccaattgaggcactaggtcaatatttatggatgtgtggtgcccctcaaagtatgagacatgacgaaaatcgtttttgtagatcaacatatacgcgcagtaagaagtttagtgaagtattgttaagtgtcaataaacttgcagcagatattattaagccattagatcctgaatttagtctcaccattttagtctcatttagtccctaaattgccaaacagtgggactaaaatagagactaaacttttttagtctctagtctctaaagaggtgactaaaagagactaaactatataaattttaccttttgtctcttctttatttcagtaattttggtcctcctatggtttcatttaatgcgttttgaataattttagtctttagaaccaaacggagtagagactaaactgtagtattctaactaaactttaatccctaaactaaagaaatcaaacgggtccttagtcactaaactttagtcagcccagaacaaacggggcctaagtgtttacttgtgcttattcttcaaatagaaaagtaaaaaatcaccttgaaaaccaattgtacctagtgcagagaggtaaataaaacgacattgagagttaaaggacgaaatttgctcggtttcaaacttcgaatagaaaacttagatggcaattatatacaccagtttagaatccttcttttttattgtgtttggtttatggagtcacccaatcattttgatgcatcataagttcattccatcaattttggtggaatcaactcacttctcacgtatatactaattattagcttatctctacaactattaagactctaatgtagaccactaccgtgcctccgctcgcgcttcgcatgcaacaacctccctgcatgcaagccgcatgcaagcgacgcaagttgctcaacctccactgcatgcaagccgcatgcaaaccacatgcaagccacatgcaagccgcaagctgctgcagaacctgcatgcaagccgagaagctgctgcatgcaagcatcgtgtctccctccactcatgaaacgtcgtggaaattgaagggggctggcctggtggctggctgttgtccgtctgtccctctgctacaagcctgtagtgctgctcagctgttgtcggctcgatcgattcccctacctgggcagcgctagccggctagccctatggactcgaggcctgattcgcaatcttactcttacccactggtcgcagccgctggtagcatcgtacaatctacaatttccagttcgagaataaaatcagctagataaaaaccacaaatccaacaaaatctaatatgtactcgcacaaaaacaattaccatatatactttttaatttaggtcatcaaccactatgcactaaggaacactaagcaagcactgacggggctgggtgggaggaacctagcgaggcgccacccatcgtgtttcctctgaggctctgcgatcgtcgttgtcttgttgcatgcactgaggctgaacgcgctgctgctactcctctgcctgtgcgcgttccccgccccggcgcggtcgcagaacacgaccatggccacgaccgcgcaggcgtccgtcgaggggttcaactgcaccgccaaccgcacgtacccatgccaggtgtacgcgctctaccgcaccggcttcgcgggcgtgccgctcgatctcgcggccatcggtgacctctttgccgtcagctgcttcatggtcgcgcacgccaacaacctctccacgacggccgcgctggccaacggacagccgctgctcgtgccgctctagtgcggctgcccctcccggtaccccagctcgtacgcgccgatgcagtaccagatcgactcggggacacctactggatcgtctccactaccaagctgcagaacctcatgcagtactaggccgtggagcgcgtgaaccccacgctggtgcccaccgtcctcgacgttggcaccatggtcacgttccccgtcttctgccagtgcccggccgccgccgacaatgccacgacactcgtcacctacgtcatgcagcccggggacacgtacgtgtctgttgccgccgccttctccgtcgcccatccacagtgaaagacacgatgcggcggccgcctgtgcatgcactgaagttgcgcgctgctgctgctcctctgcctatgcgtgttccccgccccgatgcggtcgtagaacacgaccacagccacgaccaaatttattcaactagatggagttgaaggctgaaggtacgacgggtgataaaacgatatatgatttgtgcatgaatatgtgcttcagcttgtgcaacataacaggttagaaatttaactatctgcgtgcggttattaatgtcttagaaattttaactctcgtggcaacgcacgggcacatatctagtaagaaatgaagtggtgatggataaacttattctatttaacaaaccaaataaaaatatgagaagtgagaagaagatagatcacgtcattgctcaaaccaaacaaccgaacgaaacccgtcgatcagaacccgtgctacccataatgaaagcccatgggcctaaaggcctagcccaccgttcttctcgtgggacccacacggagaaccgctctgccgacaggtggggtgacccgagccggccaggaaccaacgcgccgcgccgcccatttgcggcgtgcataccttttatttatcggctacccaacatggcagacagctgggccctggcacgcattattgccccacttgccaggcgaagtgagctgcccaccgcgctcctccgcgtcgctttgcgattttctggctcgttcccaccgctgcaacccggaacttgacgcagtctctccatgacaacatggcccacaagcgtgggtcccctactcacggactgtacacatatgaggtagagcaataccgcgtacttacagcccactccttacttttttagtttctgtctagttttgatgaggaacggaaacggaagcgccgctcaccaaggcgtaatgtctacccttggcctccttctcactgacatccgggtctcccgcagcacccacatgggtagaggaaaaacagtgggcaggtcgttctattctacggaaacggaacccacagcatcgtaggcgaaactgacaccctgtcccccgccttccccgacctcctcttcttctcctcgccggcccccgctatggtctcatccgtgtgttccccacctaaccccaccccccaatccgcc